(19) United States
(12) Patent Application Publication
(10) Pub. No.: US 2021/0113391 A1
(43) Pub. Date: Apr. 22, 2021

(54) UNDERPANTS

(71) Applicant: Vivian Barbara CUEVAS DUMOIS, Liège (BE)

(72) Inventor: Vivian Barbara CUEVAS DUMOIS, Liège (BE)

(21) Appl. No.: 16/498,441

(22) PCT Filed: Mar. 23, 2018
(86) PCT No.: PCT/EP2018/000110

§ 371 Date: Sep. 27, 2019

Related U.S. Application Data
(60) Provisional application No. 62/531,629, filed on Jul. 12, 2017.

(30) Foreign Application Priority Data
Mar. 27, 2017 (DE) 10 2017 002 942.3
Jul. 27, 2017 (DE) 10 2017 007 014.8

(51) Int. Cl. A61F 13/74; A61F 13/49; A61F 13/53

(52) U.S. Cl. CPC A61F 13/74; A61F 13/53; A61F 13/4906; A61F 13/49006

(57) ABSTRACT

The invention concerns a pair of trousers having an absorbent fleece, the fleece being incorporated into the pair of trousers and being capable of having a tampon.

The invention concerns a pair of trousers with an absorbent fleece in the crotch area which is firmly attached to the trousers. Advantageous further trainings are the subject of the subclaims.

The invention relates in particular to a women's slip. A bandage can be inserted into a panty. As an alternative, women's briefs with an absorbent layer are also known.

As a rule, a single-seamer, also called a gusset, is provided in a panty. The gusset usually looks like a small bag. However, this piece of fabric in the slip is usually not a pocket, but has several functions.

Gussets are used in the crotch area for the following reasons, whereby several can complement or exclude each other:

For better hygiene, a gusset is widely used as an additional partly undyed fabric layer or plating in the genital area on the inside of women's underpants. The words hygiene gusset, crotch lining, slip, lingerie gusset or simply gusset are commonly used as designations. A gusset can also be a combination of the main material, such as often used cotton, as a cotton gusset. Silk, satin or microfibre are also used. Some of these are still provided with an antibacterial effect and are intended to absorb excrements such as normal vaginal discharge or sweat and let them dry by evaporation and thus be more pleasant on the skin in the intimate area. Skin irritations, fungal and urinary tract infections should also be reduced. The upper material above can also be a net fabric to improve the breathability, which is criticized with an upper material made of synthetic fiber. German also refers to the hygienic gusset in net optics. In English, this is more specifically referred to as “Ventilated cotton gusset”. As an inexpensive alternative also only as a pure net, which itself often consists only of synthetic fibre. Even when hydrophilic fabrics are used, the moisture absorption is criticized as insufficient. Fabric thicknesses range from two fabric layers to net structure. The pubic cleft is usually not completely covered with underwear. In case of insufficient function or lack of this gusset, a panty liner can be glued in or additional underwear can be worn. Underwear without a second layer of fabric or plating can also be thicker or provided with honeycomb structures.

A sewn-in briefs protects against the rough fabric of the underwear and also against other external influences, such as jeans.

It also improves the vaginal flora in ladies because it absorbs the effluent and allows it to dry faster. Therefore, the gusset is mostly made of cotton, because it is particularly absorbent. This prevents a humid climate that could promote the formation of bacteria.

In the context of this application, gussets are also used for men's underpants. These inserts are located in the front area of the underpants, in which an operation can also be planned. They have the same purpose as corresponding sewn-in underpants. However, they are not exactly in the crotch area but slightly higher in the front area of the trousers.

The invention is based on the task of further developing such trousers.

In the present case, the trousers are underpants and preferably a thong. This ladies briefs is made of textile material in the usual form of a ladies briefs, whereby only in the crotch area an insert is provided which as an absorbent fleece is limited only to the crotch area. In contrast to a nappy, the invention covers only the crotch area and in particular only a maximum of 50% and preferably less than 30% of the surface of the trousers. In the case of a thong, only the absorbent fleece with narrow fastening straps can essentially form the trousers.

Especially when the trousers or briefs consist only of the gusset and ribbons or narrow strips of fabric, the function of the trousers lies above all in the function of the gusset, which is only held by the rest of the trousers and possibly embellished with lace etc.

If the gusset is filled with an absorbent material, the gusset may have a thickness of more than 0.5 cm, preferably more than 1 cm or even more than 1.5 cm when unpressed. This makes it possible to arrange a lot of absorbent material in the gusset. Especially in such a case it is advantageous if the gusset has a curved surface with a raised area in the middle between the leg openings, which is particularly absorbent due to the greater thickness in this area. In addition, this area can be convex so that it can interact with a concave surface of the user's body when wearing trousers. In the simplest case, this can be a regional elevation, a bulge extending in the longitudinal extension of the gusset, or even a tampon-like elevation.

This insert is preferably made together with the trousers and is distributed in one piece with the trousers. If the insert is soiled, the absorbent fleece insert must be disposed of or washed together with the textile trousers.

For this purpose, the panties can be opened on both sides and pulled out, for example without having to take off a pair of overpants, by pulling the panties out between the legs.

Firmly connected means that the absorbent fleece is not loose in the trousers but is not connected to the trousers in a displaceable way. In extreme cases, this is a pair of trousers with a one-piece fleece. But this can also be a fleece sewn into the trousers. This can be a fleece inserted into a pocket in the trousers and removable from the pocket, or a fleece that is firmly attached to the trousers with a Velcro fastener, for example. In all cases the trousers have been specially prepared for the fleece.

For opening, Velcro fasteners, snap fasteners, hooks and eyelets or trouser buttons can be provided on the side of the slip on opposite sides.

The briefs can also be designed as lace briefs or thong briefs.

This makes it possible to change the trousers without having to take off a pair of overpants or shoes. Thus a new pair of trousers or a new pair of small briefs can be carried in a suitable small bag, which can easily be unpacked and put on. The worn trousers can then be placed in the small bag and disposed of or washed later.

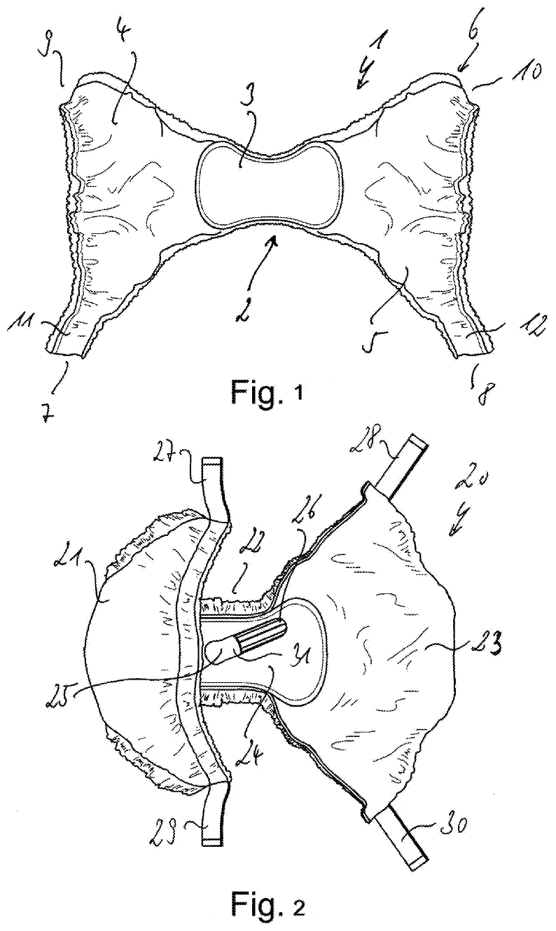

It is particularly advantageous if the inlay of the trousers also has an absorbent element such as a tampon attached to the inlay, which is attached to the absorbent fleece and can move in relation to the fleece. An advantageous example without tampon is shown in FIG. 1 and an example with tampon is shown in FIG. 2.

It is advantageous if the absorbent fleece has liquid conductors. These can extend like veins of a leaf of a deciduous tree in a fan shape (ginkgo or orchid) or can also branch (maple).

These veins make it possible to transport the liquid away from the tampon. You can also transfer liquid to the fleece to use a larger area of the fleece or even the entire fleece to absorb liquid. For this purpose, the fleece may contain superabsorbents or other liquid-binding substances.

The distribution of the liquid also helps to quickly detect contamination of blood.

The trousers or at least the fleece are designed as a disposable product and preferably only for use until the first contamination at the latest.

Advantageous further training is the subject of the subclaims. An example of the execution of the training according to the invention is shown in the drawing and will be described in more detail below.

It shows

FIG. 1 a top view of an open panty,

FIG. 2 an open ladies briefs with tampon and the

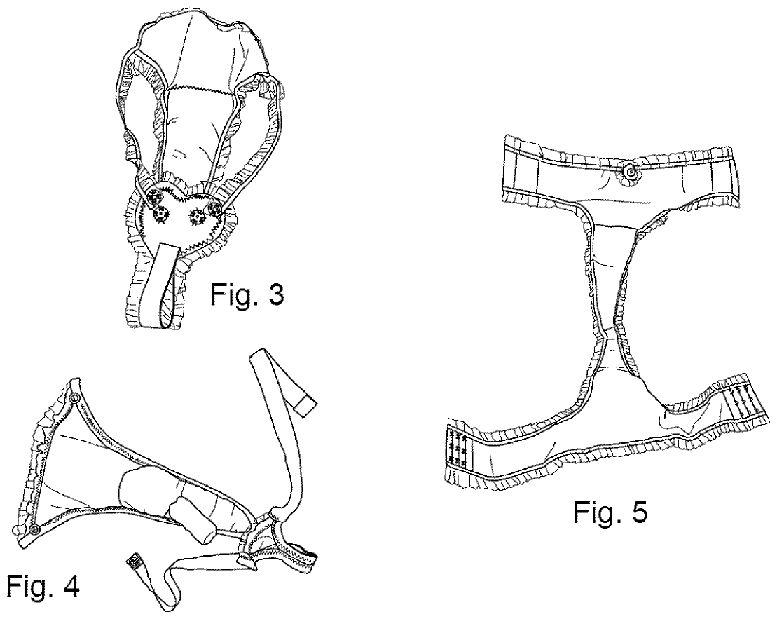

FIGS. 3 to 5 further examples of execution.

FIG. 1 shows a pair of trousers 1, in particular a pair of underpants or a ladies briefs, with an absorbent fleece 3 in the crotch area 2. This absorbent fleece 3 is firmly bonded to the other trousers 1 and worked into the women's briefs in the manner of a gusset. These trousers 1 are designed as women's briefs and have a front part 4 and a back part 5, which are joined together in crotch area 2. In the waist area, in which the front part 4 and the back part 5 lie against the hip, the trousers have on two opposite sides 7, 8 and 9, 10 fasteners 11, 12 to connect the sides 7 and 8 with each other with the fastener 12 and to connect the sides 9 and 10 with each other speaking with a fastener (not shown).

The absorbent fleece 3 is worked into the trousers 1. The trousers can also have the shape of a thong and in the example they are designed as disposable trousers suitable for absorbing liquid excreta (menstruation, vaginal discharge, sweat, etc.).

FIG. 2 shows an execution example which essentially corresponds to the execution example in FIG. 1. Here a front part 21 of a trouser 20 is connected in crotch area 22 with a rear part 23 and the fleece 24 is connected via a flexible connecting piece 25 with an ergonomic tampon 26 for the absorption of liquid excreta. The tampon 26 is thus flexibly connected to the absorbent fleece 3 as an absorbent element. This makes it possible to position the area of the fleece 3 relative to the vagina of the lady wearing the trousers 1.

The front part 21 is connected to the rear part 23 on the one hand via fasteners 27 and 28 and on the other hand via fasteners 29 and 30. The flexible connecting piece 25 has a sleeve 31 at its end remote from the absorbent fleece 3, into which the tampon 26 can be partially inserted.

The other designs shown in FIGS. 3 to 5 show how the absorbent fleece can be restricted to a substantially acute-angled, triangular area in the crotch area, while the trousers can be designed in different ways as ladies' briefs. The connection between the front and back can be similar to a brassiere with parallel eyelets, which are engaged by a hook strip. The design examples show that a wide variety of women's briefs can be produced, which on the one hand offer little fabric area and on the other hand in the crotch area an absorbent fleece area which is designed to absorb liquids. This crotch area can be equipped with special highly absorbent materials, such as superabsorbents, in order to optimise the absorbency of this area, while the other areas of the lady's briefs can be visually appealing in different forms.

For example, the women's briefs can have several layers arranged where the bandage is normally inserted. A layer is provided all the way inside, which provides a surface that feels dry to the touch. Then there is an antibacterial layer, then there is an absorbent layer that can absorb liquid, and the outermost layer is a repellent layer that prevents the transition from liquid to a pair of trousers worn over it. The briefs generally make it possible for incontinence or a lady to manage without a bandage, insert or diaper during her days. The lateral opening of the trousers allows a quick change of the trousers. This means that a smaller area is sufficient for the absorbent material, which can be limited to the gusset of the trousers.

A V-shape of the absorbent fleece enables a particularly good adaptation to the female anatomy in the genital area through optimal coverage of the genital area including the vulva and counteracts the rolling up of the fleece through movement. Such a V-shaped fleece can be formed with or without a tampon. The fleece preferably has a padding.

For single use, the trousers may be designed as disposable trousers. If the trousers have a tampon or a tampon-like suction element, this can have pores for better ventilation of the genital area. A flexible attachment of a tampon allows automatic adaptation to different anatoms and provides better wearing comfort during movements.

The absorbent fleece can also have an oval shape.

The gusset can also be dumbbell-shaped.

The excrements can be centrally absorbed by the fleece with such trousers. These trousers are particularly suitable for women of menstrual age.

1-12. (canceled)

13. A women's slip (1) comprising:
a front part (4) and a rear part (5), which are connected to one another in the crotch region (2); and
an absorbent fleece (3) in the crotch region (2);
wherein the front part (4) and the rear part (5) are detachably connected to one another in the waist region (6) on two opposite sides (7, 9 and 8, 10) like a diaper, the absorbent fleece (3) being firmly connected to the slip (1) and being incorporated into the slip (1).

14. The slip according to claim 13, wherein absorbent fleece (3) is connected to a tampon (26).

15. The slip according to claim 14, wherein the slip comprises a flexible connecting part (25) which is bonded to the fleece (3) and the tampon (26).

16. The slip according to claim 15, wherein the connecting part has a sleeve (31) into which the tampon (26) is partially inserted.

17. The slip (1) according to claim 13, wherein the absorbent web (3) covers at most 50% of the surface of the slip.

18. The slip (1) according to claim 13, wherein the absorbent fleece (3) is an incontinence pad firmly connected to the slip (1).

19. The slip (1) according to claim 13, wherein the absorbent nonwoven (3) has a substantially triangular basic shape.

20. The slip (1) according to claim 13, wherein the absorbent web (3) comprises liquid conductors.

21. The slip (1) according to claim 20, wherein the liquid conducting wires begin at a tampon (26) and extend into the fleece (3).

22. The slip (1) according to claim 20, wherein the liquid conducting veins branch like veins of a leaf and taper towards their end.
